(19) United States
(12) Patent Application Publication
(10) Pub. No.: US 2023/0277135 A1
(43) Pub. Date: Sep. 7, 2023

(54) SYSTEM FOR INTRAORAL MONITORING AND RELATED METHODS THEREOF

(71) Applicant: Dianyx Innovations, LLC, Westerville, OH (US)

(72) Inventors: Robert Kibler, Columbus, OH (US); Seyed Nabavi, Montreal (CA); John Cogan, Blackrock County (IE); Asim Roy, Dublin, OH (US); Brandon Canfield, Columbus, OH (US); Collin Emerick, columbus, OH (US)

(73) Assignee: Dianyx Innovations, LLC, Westerville, OH (US)

(21) Appl. No.: 18/177,391

(22) Filed: Mar. 2, 2023

Related U.S. Application Data
(60) Provisional application No. 63/315,843, filed on Mar. 2, 2022.

(51) Int. Cl. A61B 5/00; A61B 5/11; A61B 7/00; A61B 5/0205; G06F 21/60; G16H 40/67

(52) U.S. Cl. CPC A61B 5/682; A61B 5/0022; A61B 5/1118; A61B 7/003; A61B 5/0205; G06F 21/602; G16H 40/67; A61B 2560/0214; A61B 5/02416

(57) ABSTRACT

An intraoral monitoring device includes a flexible substrate, a plurality of sensors, a rechargeable battery, a device module and a communication module. The plurality of sensors senses a plurality of health parameters from an associated user, in real time, to generate a plurality of sensed signals. The device module includes a memory, a controller and a segregation module. The memory stores various health parameters and their respective ranges, various health reports of the user, and basic details of all users. A controller is configured to generate sensed data and the segregation module is configured to segregate the data corresponding to the plurality of health parameters associated with the users. A communication module is configured to enable data communication to and from the intraoral device.

### TECHNICAL FIELD

The present disclosure relates to the field of intraoral monitoring devices and related methods. More specifically, the disclosure relates to an intraoral monitoring device having capabilities of measuring physiological and cardiorespiratory parameters in a user in real time. The device collects user health and sleep data by measuring these parameters from the oral cavity of the user, which can be used for diagnosis and treatment purposes.

### BACKGROUND

The background information herein relates to the present disclosure but is not necessarily prior art.

Current photoplethysmogram (PPG) sensing is an optically obtained plethysmogram that can be used to measure the different components of sleep stages based on saturation of peripheral oxygen (SpO2) and cardiorespiratory measurements. For instance, raw light signals from the sensor are mapped to SpO2 and cardiac activities. A time series of these readings can then input into a machine learning algorithm that has previously been trained and a subject's sleep measurements are output.

The monitoring of muscle activity plays an important role in the study of sleep stages. For example, the stage of rapid eye movement (REM) during sleep can be accurately determined based on the activity level or tone of the facial muscles. In some systems, electromyogram (EMG) techniques measure muscle activity. However, EMG generally requires at least three electrodes that must be placed on the skin of subject at an optimal distance from each other. Further, in EMG, the action of muscles produces electrical signals, hence, the EMG electrodes measure this produced electrical signals from the surface of the skin. Consequently, the amplitude of measured electrical signals by the EMG determines the muscle activity. Conventionally, the EMG approach is used to monitor masseter muscle activity to diagnosis bruxism. It should be noted that to acquire a high-quality EMG signal, an impedance-matching layer, i.e., an electrolyte gel, is typically deployed between the skin and the electrodes. In EMG, however, output signal is significantly impaired in the presence of sweat or moisture. Therefore, it typically cannot be implemented in wet anatomical positions, such as the oral cavity. An alternative approach is to use a single channel PPG sensor. It is shown that action of muscle changes their volumes, while this change in muscle size can be monitored by the PPG sensor. Thus, a PPG sensor can be positioned in contact with the masseter muscle. Therefore, any change in the size of this muscle due to its action can be captured based on its impact on the captured PPG signals.

Further, the use of MAD (mandibular advancement device) and CPAP (continuous positive airway pressure) machine is one of the most common treatments for sleep apnea. But the use of these technologies does not provide data storage, data communication functionalities. The maintenance of CPAP machines is also very costly and requires substantial resources and time.

To overcome the challenges to physiological and environmental monitoring presented in conventional systems, systems and methods for intraoral monitoring is leveraged in a digital medium environment. In order to mitigate the challenges of inaccurate physiological and environmental monitoring experienced in typical techniques, an intraoral device is introduced that is placed within an oral cavity and is able to accurately measure various physiological and environmental phenomena.

There is, therefore, felt a need to a need to develop an intraoral monitoring device that monitors user's health and that alleviates the aforementioned drawbacks.

### SUMMARY

Some of the objects of the present disclosure, which at least one embodiment herein satisfies, are as follows:

An object of the present disclosure is to provide an intraoral monitoring device to monitor, store and analyze substantial information related to the health condition as well as sleep architecture of the user.

Another object of the present disclosure is to provide an intraoral monitoring device that includes sensors embedded in a flexible substrate capable of measuring multiple cardiorespiratory and physiological parameters in real time from the oral cavity of the user.

Yet another object of the present disclosure is to provide an intraoral monitoring device that can be bent to conform to the shape of dental arch (i.e., the curved structure that contains the teeth in the upper or lower jaw) of the user. In this object, the intraoral monitoring device is placed inside the oral cavity of the user (e.g., the exterior of the maxilla (upper jaw) or mandible (lower jaw)).

Yet another object of the present disclosure is to provide an intraoral monitoring device that is portable and light weight.

Yet another object of the present disclosure is to provide an intraoral monitoring device which can store, analyze and segregate the plurality of health parameters of the user.

Yet another object of the present disclosure is to provide an intraoral monitoring device that is easy to maintain and is user friendly.

Yet another object of the present disclosure is to provide an intraoral monitoring device that can detect the health-related parameters of the user and communicate the same to a user's mobile application.

Other objects and advantages of the present disclosure will be more apparent from the following description, which is not intended to limit the scope of the present disclosure.

The present disclosure envisages an intraoral monitoring device. The intraoral monitoring device includes a flexible substrate, plurality of sensors, a rechargeable battery, device module and communication module.

In an aspect, the flexible substrate enables the intraoral device to be curved according to the curvature of a wearer, which promotes contact between the sensors and the wearer's skin which further provides accurate measurement signals. Further, the use of a flexible substrate enables the intraoral device to be designed and fabricated and then attached to a wide range of oral appliances, including but not limited to dental devices, mouthpieces and bite guards, regardless of their geometry, due to the conformability of the design of the intraoral device.

In an aspect, the plurality of sensors includes one or more PPG sensor, a motion sensor such as an accelerometer or a gyroscope, a pressure sensor, a temperature sensor, a pH sensor, an acoustic sensor, and a dielectric sensor used for analysis of user saliva and/or measuring tongue position during sleep. The PPG sensors are used for detecting cardiorespiratory parameters, muscle activity and/or attributes of blood volume, such as in the microvascular bed of tissue. They are positioned to measure muscle activity of the lateral pterygoid, buccinator, and masseter muscle from inside an oral cavity. The sensor data from the plurality of sensors is combined to enable accurate inference of sleep-related conditions. It can be utilized to monitor various sleep stages.

In an aspect, the PPG signals obtained from the PPG sensors are robust to moisture.

In an aspect, the battery represents a power source for the intraoral device and can include a single battery or an array of batteries.

In yet another aspect, the device module includes memory, controller and the segregation module.

The sensed data from the plurality of sensors is stored in the memory and sent to another device via a communication module. If a connection between the intraoral device and another device is interrupted, the sensor data can be temporarily stored in a memory of the device module and/or in an on-board flash memory. Once a stable connection is re-established between the intraoral device and a receiving station, real-time data transmission can be resumed.

In an embodiment, the device module is configured to analyze the real time sensed data by employing machine learning techniques such as Gradient boosted techniques, Decision tree techniques and Logistic regression techniques to evaluate emergency admission cases with prediction and analysis using Information Communication Technology (ICT) techniques.

In another embodiment, the controller is configured to employ energy efficient FoG based IoT network techniques to monitor the user's health conditions.

In still another embodiment, the memory is communicatively coupled to a database. The database is configured to store various health parameters of the user.

In an aspect, the system for intraoral monitoring device includes an intraoral device, a docking station, a client device, a device management platform (DVMP), a data management platform (DMP), and a remote monitor system (RMS). The intraoral device, docking station, client device, DVMP, DMP, and RMS are interconnectable in various ways such as via connectivity to a network and/or via direct device-to-device connectivity.

Advantageously, the communication module cooperates with the controller to transmit the data to the cloud-based server for data storage, monitoring and segregation based on a user defined algorithm using machine learning techniques.

Advantageously, the plurality of health parameters is selected from a group consisting of saturation of peripheral oxygen, temperature, pressure, motion, sound, pH, apnea hypopnea index (AHI) and a combination thereof.

Advantageously, the controller can be implemented as one or more microprocessors, microcomputers, microcontrollers, digital signal processors, central processing units, state machines, logic circuitries, and/or any devices that manipulate signals based on operational instructions.

### BRIEF DESCRIPTION OF THE DRAWINGS

An intraoral monitoring device disclosed in the present disclosure will now be described with the help of the accompanying drawing, in which:

FIG. 1 is an illustration of an environment in an example implementation that is operable to employ systems and methods for intraoral monitoring as described herein.

FIG. 2 depicts an example implementation of the intraoral device including different instances of the sensors and placed within an oral cavity in accordance with implementations described herein.

FIG. 3a depicts an example implementation of the docking station in accordance with implementations described herein.

FIG. 3b depicts the docking station with the door in an open position relative to the housing.

FIG. 4 depicts a system for implementing aspects of systems and methods for intraoral monitoring.

FIG. 5 depicts a method for intraoral monitoring and utilizing sensor data obtained as part of intraoral monitoring in accordance with one or more implementations.

FIG. 6 depicts an example method for security attributes as part of intraoral monitoring in accordance with one or more implementations.

FIG. 7 depicts graphs that illustrate intraorally measured PPG signals at red and IR wavelengths in accordance with one or more implementations.

FIG. 8 depicts graphs that illustrate that illustrate estimated HR, SpO2, and RR from the intraorally measured PPG signals in accordance with one or more implementations.

FIG. 9 depicts a graph that illustrates intraorally monitoring masseter muscle activity by using a PPG sensor in accordance with one or more implementations.

FIG. 10 depicts a graph that illustrates muscle activity measured by an intraoral device in accordance with one or more implementations.

FIG. 11 illustrates an example system that includes an example computing device that is representative of one or more computing systems and/or devices that are usable to implement the various techniques described herein.

### DETAILED DESCRIPTION

Embodiments of the present disclosure will now be described with reference to the accompanying drawing.

Embodiments are provided to thoroughly and fully convey the scope of the present disclosure to the person skilled in the art. Numerous details are set forth, relating to specific components, and methods, to provide a complete understanding of embodiments of the present disclosure. It will be apparent to the person skilled in the art that the details provided in the embodiments should not be construed to limit the scope of the present disclosure. In some embodiments, well-known processes, well-known apparatus structures, and well-known techniques are not described in detail.

The terminology used, in the present disclosure, is only to explain a particular embodiment and such terminology shall not be considered to limit the scope of the present disclosure. As used in the present disclosure, the forms “a”, “an”, and “the” may be intended to include the plural forms as well, unless the context suggests otherwise. The terms “comprise”, “comprising”, “including”, and “having”, are open-ended transitional phrases and therefore specify the presence of stated features, elements, modules, units, and/or components, but do not forbid the presence or addition of one or more other features, elements, components, and/or groups thereof. The particular order of steps disclosed in the method and process of the present disclosure is not to be construed as necessarily requiring their performance as described or illustrated. It is also to be understood that additional or alternative steps may be employed.

As used herein, the term “and/or” includes any and all combinations of one or more of the associated listed elements.

The terms first, second, third, etc., should not be construed to limit the scope of the present disclosure as the aforementioned terms may be only used to distinguish one element or component from another element or component. Terms such as first, second, third, etc., when used herein do not imply a specific sequence or order unless suggested by the present disclosure.

Current photoplethysmogram (PPG) sensing is an optically obtained plethysmogram that can be used to measure the different components of sleep stages based on saturation of peripheral oxygen (SpO2) and cardiorespiratory measurements. For instance, raw light signals from the sensor are mapped to SpO2 and cardiac activities. A time series of these readings can then input into a machine learning algorithm that has previously been trained and a subject's sleep measurements are output.

To overcome the challenges to physiological and environmental monitoring presented in conventional systems, systems and methods for intraoral monitoring is leveraged in a digital medium environment. In order to mitigate the challenges of inaccurate physiological and environmental monitoring experienced in typical techniques, an intraoral device is introduced that is placed within an oral cavity and is able to accurately measure various physiological and environmental phenomena.

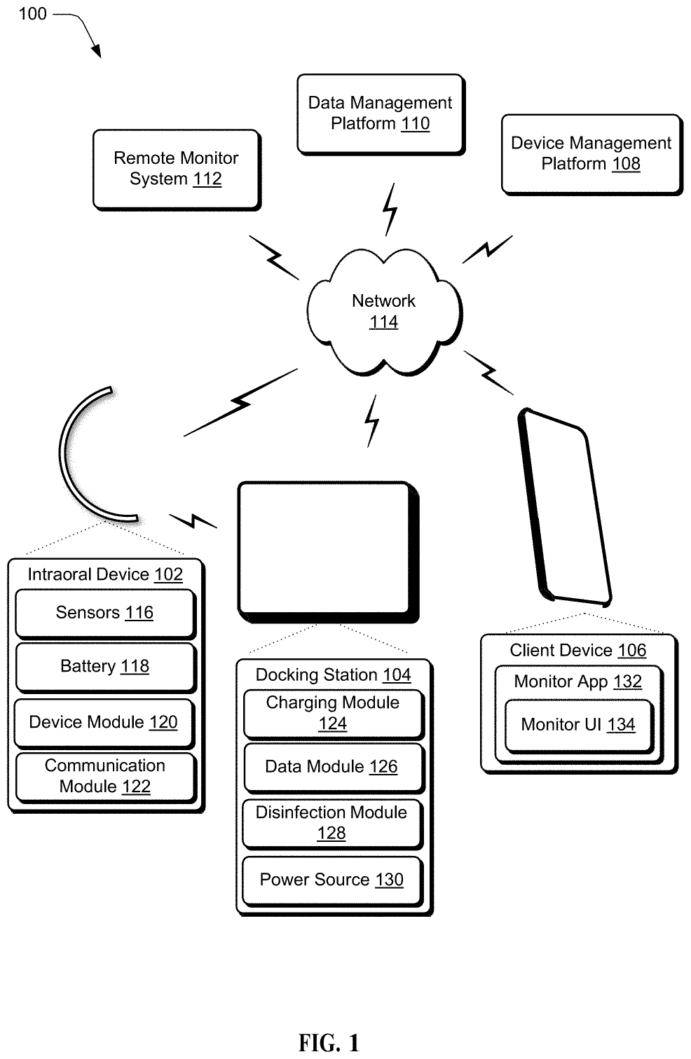

To alleviate the aforementioned shortcomings of the conventional intraoral device, an intraoral monitoring device is now being described with reference to FIG. 1.

In the present disclosure, various types of users are described that can participate in aspects of systems and methods for intraoral monitoring. The following are examples of such users:
1) Unique User—a consenting individual assigned and confirmed to access data collected by an intraoral device. A unique user, for instance, may share, assign access, allow other users (e.g., permissive users, clinicians, organizations, third parties, etc.) to access, evaluate, share, view, and/or distribute information collected, processed, and analyzed according to the described techniques.
2) Permissive User—a consenting individual, application, program, organization, and/or platform assigned, permitted by a user (e.g., a unique user) to access data collected by an intraoral device.
3) Clinician(s)—a consenting party, or parties, indicated for access to user data in order to view, evaluate, and/or manage the accessed data for purposes including but not limited to ensuring usage and compliance, therapeutic efficaciousness, disease management and improvement, monitoring behavior(s) related to sleep and wake, productive analysis, and/or other purpose related to health and wellness of a user of an intraoral device. A clinician may interact with a user in various ways, such as in-person and/or remotely via an approved application and/or platform and in a secure manner.
4) Third Parties—a consenting party, or parties, indicated for access to user data in order to view, evaluate, and/or manage the accessed data for purposes including but not limited to ensuring usage and compliance, therapeutic efficaciousness, disease management and improvement, monitoring behavior(s) related to sleep and wake, productive analysis, and/or other purpose related to health and wellness of a user of an intraoral device. A third party may interact with a user in various ways, such as in-person and/or remotely via an approved application and/or platform and in a secured manner.

According to various implementations, the sensing platform described herein comprises a plurality of sensors, e.g., PPG, pressure, temperature, accelerometer, pH, acoustic, and dielectric, while each individual sensor measures a specific physiological parameter. The output data obtained by each individual sensor from an associated anatomical location, e.g., the oral cavity, in combination with information from other sensors, produces a robust data set. Hence, by using an analytical equation and/or machine learning technique, the sensor data can be classified to accurately identify different stages of sleep. As such, reports generated using the sensor data can be used for various purposes, such as monitoring and diagnostic purposes.

### Example Environment

FIG. 1 is an illustration of an environment 100 in an example implementation that is operable to employ systems and methods for intraoral monitoring as described herein. The present disclosure envisages system and method for intraoral monitoring device 102 for monitoring, storing and analyzing the substantial information on the plurality of health condition as well as sleep stage of the user.

The environment 100 includes an intraoral device 102, a docking station 104, a client device 106, a device management platform (DVMP) 108, a data management platform (DMP) 110, and a remote monitor system (RMS) 112. According to various implementations, the intraoral device 102, docking station 104, client device 106, DVMP 108, DMP 110, and RMS 112 are interconnectable in various ways further to implementations described herein, such as via connectivity to a network 114 and/or via direct device-to-device connectivity. The network 114 can be implemented in various ways, such as a wireless network, a wired network, and/or a combination of wired and wireless networks and is implemented via any suitable architecture. Examples of the network 114 include the internet, a wide area network (WAN), a local area network (LAN), a mesh network, and combinations thereof.

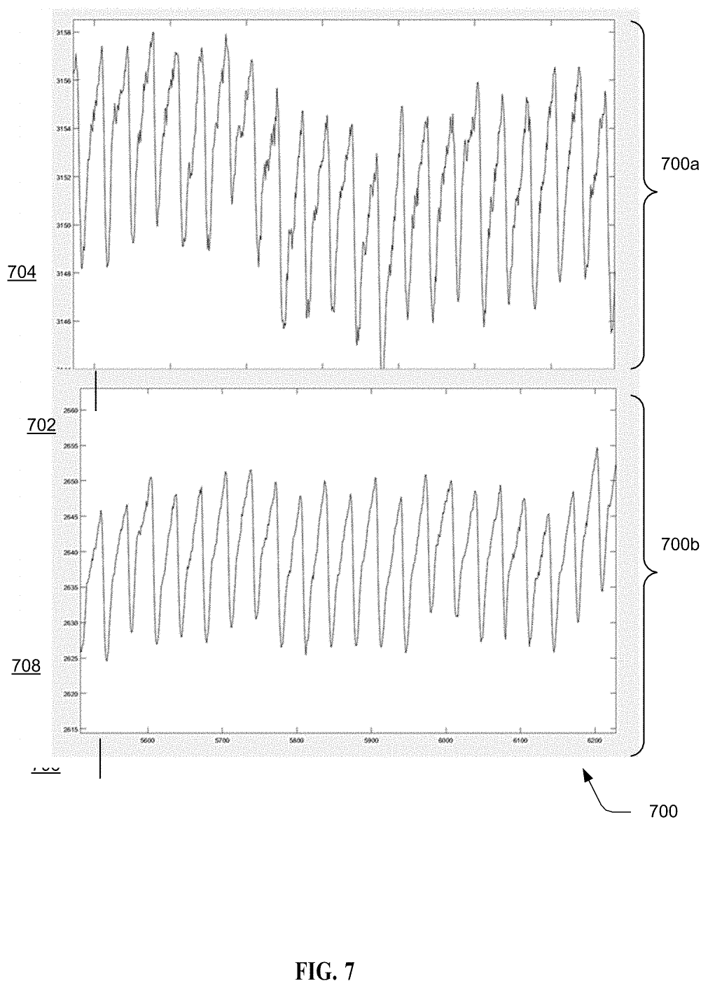

Examples of devices that are used to implement the docking station 104, client device 106, DVMP 108, DMP 110, and RMS 112 includes a desktop computer, a laptop computer, a mobile device (e.g., assuming a handheld configuration such as a tablet or mobile phone), a server device, and so forth. Additionally, the client device 106, DVMP 108, DMP 110, and RMS 112 can be implemented using a plurality of different devices, such as multiple servers utilized by an enterprise to perform operations “over the cloud” as further described in relation to FIG. 7.

The intraoral device 102 includes a plurality of sensors 116 and is configured to be positioned within an oral cavity to detect various phenomena (e.g., physiological and/or environmental attributes) such as saturation of peripheral oxygen, temperature, pressure, motion, sound, pH as well as conductivity of saliva, apnea hypopnea index (AHI), and so forth. The intraoral device 102 also includes a battery 118, a device module 120, and a communication module 122. The battery 118 represents a power source for the intraoral device 102 and can be implemented in various ways, such as a single battery, a battery array, and so forth. The device module 120 includes memory, controller and segregation module. It represents functionalities for performing various tasks for the intraoral device 102, such as data management for the intraoral device 102, such as for collecting sensor data from the sensors 116, storing sensor data, transferring sensor data to other entities, processing sensor data, segregating sensor data, transferring data to the intraoral device 102 e.g., for configuring operation of the intraoral device 102, segregating the sensed data and so forth. The communication module 122 represents functionalities for enabling data communication from and to the intraoral device 102. The communication module 122 may utilize different wireless and/or wired communication protocols, such as Bluetooth™, Near-field communication (NFC), ZigBee, and so forth.

The docking station 104 is configured to perform various functionalities for maintaining and interfacing with the intraoral device 102. The docking station 104, for instance, includes a charging module 124, a data module 126, a disinfection module 128, and a power source 130. The charging module 124 represents functionalities for charging a power supply (e.g., battery) of the intraoral device 102. The data module 126 represents functionalities for transferring data from and/or to the intraoral device 102. The disinfection module 128 represents functionalities for disinfecting and/or drying the intraoral device 102.

The client device 106 is configured to connect to the docking station 104 and/or the intraoral device 102 to provide various user functionalities. For instance, the client device 106 includes a monitor application (“app”) 132 that implements a monitor user interface (UI) 134 such as a graphical user interface (GUI). According to various implementations, the monitor app 132 receives data that is generated by the intraoral device 102, such as sensor data from various sensors that is generated by and/or stored on the intraoral device 102. Further, the monitor app 132 can enable data transfer to the intraoral device 102, such as for configuring (e.g., updating and/or repairing) functionalities of the intraoral device 102.

In at least some implementations, the monitor app 132 represents a downloadable interface such as for sending and receiving user data throughout the present system. For instance, the monitor app 132 can receive data from the intraoral device (e.g., directly and/or via the docking station 104) and transmit the data to the DVMP 108 and/or the RMS 112. Further, the monitor app 132 can receive data from the DVMP 108 and/or the RMS 112. Utilizing the monitor app 132, users can view and share data, including outside the present system in a number of categories, including but not limited to permissive users, other users of the present system, applications, platforms, clinicians, organizations, and so forth. Unique users, for instance, give the present system initial consent to collect data via the monitor app 132, and after this consent the present system initiates data collection.

The DVMP 108, the DMP 110, and the RMS 112 represent functionalities for performing various functionalities for systems and methods for intraoral monitoring described herein. For instance, the DVMP 108 represents functionalities for brokering data transfer between different entities of the environment 100 and/or for performing processing of data received from the client device 106, e.g., sensor data generated at the intraoral device 102. The DVMP 108, for instance, can receive data from the monitor app 132 and communicate the data to the DMP 110 and/or the RMS 112. Further, the DVMP 108 can receive data from the DMP 110 and/or the RMS 112 and communicate the data to the monitor app 132.

The DMP 110 represents functionalities for data aggregation (e.g., sensor data), data analysis via algorithms and other proprietary methods, report generating, data sharing, implementing machine learning attributes, data storage, and so forth. The DMP 110 also provides integration with applications, platforms, and application program interfaces (API) as well service providers such as electronic health records (EHR) systems, and blockchain systems.

Example functionalities that may be implemented individually and/or cooperatively by the DVMP 108 and the DMP 110 include:
(1) Data sharing with permissive users with appropriate permissions, data analysis using algorithms, and machine learning tasks;
(2) Responding to queries from various entities;
(3) Securing interaction with application programming interfaces (API) for approved and authorized purposes;
(4) Secure authentication for the users with appropriate permissions, such as permissive users;
(5) Ordering user data for data analysis of user data, such as nightly, weekly, monthly, and other.
(6) Executing machine learning algorithms and presenting results in accordance with user requests, such as for unique users, permissive users, clinicians, third parties, and so forth.
(7) Sending and receiving user data in encryption
(8) Exacting insights from data or lack of data from a user. Some examples include patterns in usage, changes in physiological information (heart rate, respiratory rate, oxygen desaturation, muscle activity, AHI, housing temperature, head position, tongue position, PH level, sound in decibels, and others) relative to the unique user. These insights may be used by permissive users for clinical purposes, such as to improve health outcomes.
(9) Express critical values related to physiological information (such as described above) in a manner customizable by a user for the purpose of alerts, reporting, population health comparison, titration levels of the intraoral device 102, and so forth.

The RMS 112 represents functionalities for data analysis via algorithms and other proprietary methods, report generating, data sharing, data storage, and so forth. The RMS 112 can implement a cloud-based web application accessible by permissive users, clinicians, organizations, third parties, and so forth. For instance, permissive users can interact with the RMS 112 to view, evaluate, manage, and analyze user data such as for disease management, therapeutic adherence, and efficacy, e.g., via reporting and testing measures. In at least some implementations, the RMS 112 can enable the diagnosis and treatment of sleep disorders by collecting patient health data sourced by the intraoral device 102 and allowing a provider (e.g., clinic, doctor office, etc.) to monitor the health data and provide wellness guidance and directions to the patient based upon the results. The RMS 112, for instance, enables remote patient monitoring where a patient and a practitioner may be locationally remote from one another and data obtained from an intraoral device 102 installed in a user is processed and provided to the RMS 112, where a practitioner may access and analyze the data from any suitable location.

According to various implementations, appropriate data security protocols are observed as a part of collecting and maintaining data in the environment 100 and for the life of the data within the environment 100. Examples of different data handling protocols are described below.

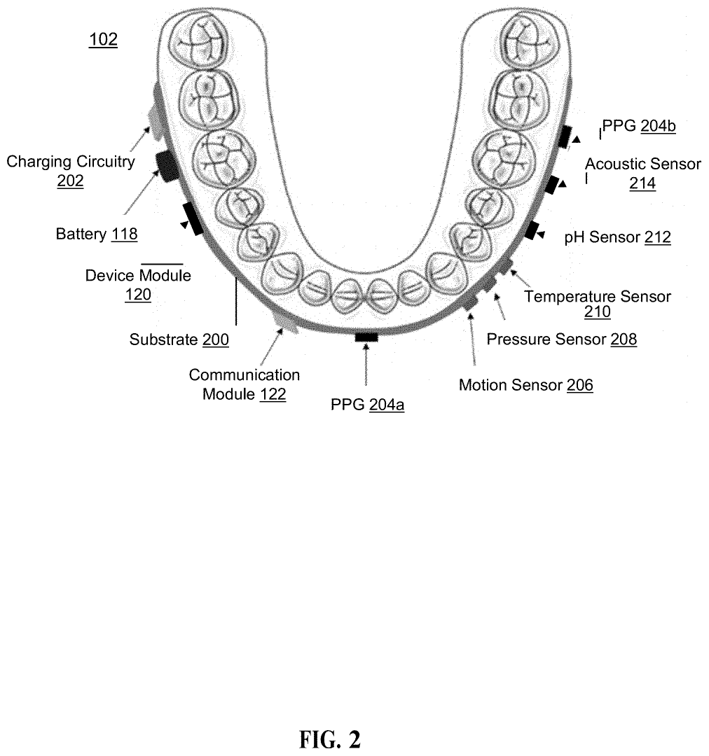

FIG. 2 depicts an example implementation of the intraoral device 102 including different instances of the sensors 116 and placed within an oral cavity in accordance with implementations described herein. The intraoral device 102, for instance, includes a flexible substrate 200 with the sensors 116, the battery 118, the device module 120, and the communication module 122 attached to (e.g., embedded within) the substrate 200. Also depicted in this example is charging circuitry 202 which is operable to enable charging of the battery 118.

The substrate 200 may be formed from any suitable material that enables placement of the various components of the intraoral device 102 as well as positioning of the intraoral device. The substrate 200, for instance, is formed from a flexible material such as Kapton™ tape and/or other flexible material, such as a flexible polymer. In at least one implementation, electrical connection between the various components of the intraoral device 102 on the substrate can be implemented in various ways, such as using silver ink. The use of a flexible substrate is advantageous such as it enables the intraoral device 102 to be curved according to the curvature of a wearer, which promotes contact between the sensors 116 and the wearer's skin and, consequently provides accurate measurement signals. Further, the use of a flexible substrate enables the intraoral device to be designed and fabricated and then attached to a wide range of oral appliances, regardless of their geometry, due to the conformability of the design of the intraoral device 102.

In this particular example the sensors 116 include a PPG sensor 204a, a motion sensor 206, a pressure sensor 208, a temperature sensor 210, a pH sensor 212, an acoustic sensor 214, and a PPG sensor 204b. The PPG sensors 204a,204b, represent functionalities for detecting cardiorespiratory parameters, muscle activity and/or attributes of blood volume, such as in the microvascular bed of tissue. In at least some implementations, each PPG sensor 204 consists of two or three source lights (LEDs) at different wavelengths, i.e., red, infrared (IR), or green, and a photodetector. Thus, the reflection/transmission of the source light from/through the skin can be measured by the photodetector. Generally, the topology of PPG signals, including but not limited to, amplitude, period, frequency, and variation, can be used to identify different stage of sleep.

In at least some implementations, one or more of the PPG sensors 204a,204b are positioned to measure muscle activity of the masseter muscle from inside an oral cavity, such as using a PPG sensor 204 positioned adjacent (e.g., fixed to) an upper molar. The PPG sensor 204a, for instance, is positioned on the substrate 200 such that when the intraoral device 102 is installed in a user's oral cavity, the PPG sensor 204a is positioned near a central incisor (e.g., teeth No 8 and 9), facing the inner skin of the upper lip. Further, the PPG sensor 204b is positioned on the substrate 200 such that the PPG sensor 204b is positioned at a location close to the molar area (e.g., teeth #1-3 or #14-16) adjacent the masseter muscle. Even with the intraoral device 102 placed within an oral cavity, PPG signals from the PPG sensors 204 are relatively robust to moisture. In at least one implementation, the effect of a wet and/or humid environment on the PPG output signals can be ignored, e.g., since light utilized by the PPG sensors 204 can be passed through a transparent medium and reflected as part of sensor operation. For instance, it is shown that action of muscle changes their volumes, while this change in muscle size can be monitored by a PPG sensor. Thus, the PPG sensor 204b can be positioned on the substrate 200 in contact with the masseter muscle. Therefore, any change in the size of this muscle due to its action can be captured based on its impact on the captured PPG signals.

In at least one implementation the PPG sensors 204 utilize at least two LEDs at different wavelengths such as red and IR or green and IR. The PPG output signals can be used for estimating heart-rate (HR), respiratory rate (RR), and SpO2. Furthermore, the PPG data (single channel or dual channel) can be used to determine different stages of sleep, such as after applying signal processing techniques (e.g., denoising, smoothing, averaging, etc.) to the PPG data.

It should be noted that the PPG sensors may extract signal from any suitable artery and in one embodiment the signal is received from inferior labial artery.

The motion sensor 206 represents functionalities for detecting motion and can be implemented in various ways, such as an accelerometer. According to one or more implementations, the motion sensor 206 represents a three-axis accelerometer usable for actigraphy. Actigraphy, for instance, monitors movement and can be used to assess sleep parameters, such as sleep-wake cycles, and body position. In the intraoral device 102, output of the motion sensor 206 can be used to determine a user's head and housing position, e.g., supine, prone, right side, left side, and so forth. Further, intraorally measured motion data can be used for sensor fusion techniques, such as for removing motion artifacts from PPG signals received from the PPG sensors 204a,204b. For instance, motion frequency determined by motion data from the motion sensor 206 is input to a digital filter such as a notch filter and is applied to PPG data to subtract a frequency of motion artifacts. The depicted position of the motion sensor 206 on the substrate 200 is presented for purpose of example only, and the motion sensor 206 can be placed at any suitable location.

The pressure sensor 208 represents functionalities for detecting pressure and can be implemented in various ways, such as a microelectromechanical system barometer, a piezoelectric gauge, a piezoresistive gauge, and so forth. The pressure sensor 208 is integrated into the substrate 200 to measure the intraoral pressure, such as for measuring airflow during inhalation and exhalation. Since pressure and volume are inversely proportional, therefore, the volume of air inside the oral cavity, which is a function of the airflow from the nasal passage, can be estimated based on the output of the pressure sensor 208. The depicted position of the pressure sensor 208 on the substrate 200 is presented for purpose of example only, and the pressure sensor 208 can be placed at any suitable location.

The temperature sensor 210 represents functionalities for measuring oral cavity temperature as well as core housing temperature. Measured housing temperature can be combined with other measured parameters and used to assess sleep quality. The depicted position of the temperature sensor 210 on the substrate 200 is presented for the purpose of example only, and the temperature sensor 210 can be placed at any suitable location.

Temperature data from the temperature sensor 210 can be utilized for various purposes. For instance, prior to entering a sleep state, a user's body temperature begins to reduce. For instance, core temperature can be reduced by 1 to 2° C. during sleep. Further, an average body temperature curve can increase slightly during rapid eye movement (REM) sleep stage.

The pH sensor 212 represents functionalities for measuring the pH (e.g., relative acidity or alkalinity) within an oral cavity. The pH sensor, for instance, continuously measures the pH level of the oral cavity, e.g., saliva pH data from the pH sensor 212 can be utilized for various purposes. For instance, a normal pH range for saliva is 6.2 to 7.6. Further, intraoral pH can decrease slowly over a sleep session, while sleeping with breathing via the oral cavity can result in a further decrease in pH over a longer period of time. Thus, measured pH levels can be used to identify breathing routes and incorporated with other sensor outputs to accurately determine sleep stages.

The acoustic sensor 214 represents functionalities for measuring audio signals. The acoustic sensor 214, for instance, represents a low power microphone that is integrated into the substrate 200 to measure the sound intensity in the oral cavity, such as the intensity of snoring from the oral cavity. Sounds data captured by the acoustic sensor 214 can be utilized for various purposes. For instance, sound data can be used to monitor the intensity and duration of a user's snoring and the user's effort to breathe during sleep. Thus, the intensity and frequency of output signals of the acoustic sensor 214 can be used to determine both of these factors.

While not shown explicitly, the sensors 116 may also include one or more dielectric sensors. The dielectric sensors may be configured to accomplish one or more functionalities such as analysis of user saliva and/or measuring tongue position during sleep.

According to various implementations, sensor data from the different sensors is combined to enable accurate inference of sleep-related conditions. For instance, combining sensor data enables different parameters (e.g., sensor output signals) to be utilized to monitor sleep stages. Further, interpolation of output signals received from a set of sensors and/or sensor data fusion provides for highly accurate characterization of health conditions, such as different user sleep stages. In at least one implementation sensor data can be used to measure AHI.

Hence, by using an analytical equation and/or machine learning technique to analyze the sensor data, the sensor data can be classified to accurately identify different stages of sleep. As such, a report generated by the RMS 112 can be used for various purposes, such as monitoring and diagnostic purposes.

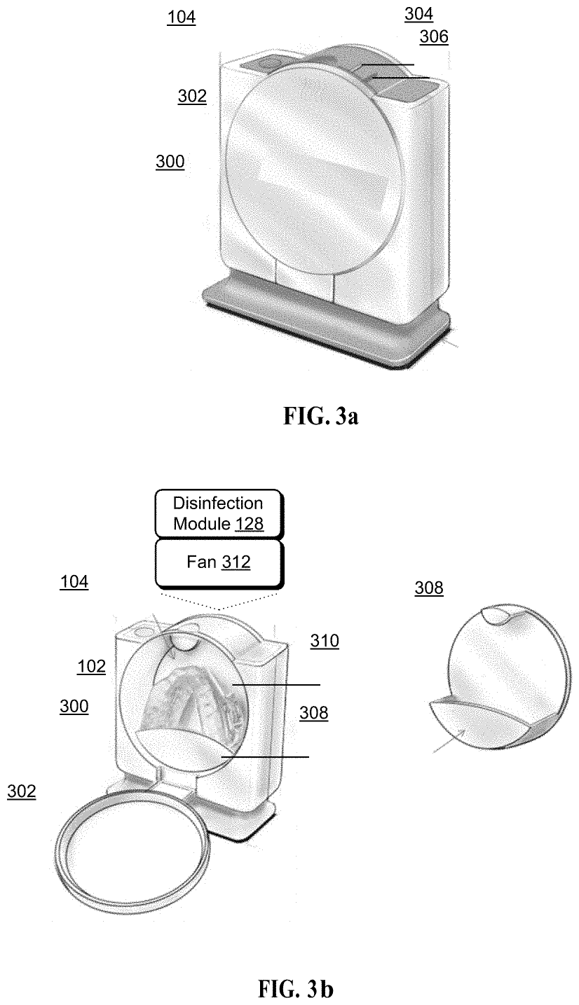

FIG. 3a depicts an example implementation of the docking station 104 in accordance with implementations described herein. In FIG. 3a, the docking station 104 is depicted in a closed position, such as with the intraoral device 102 positioned within an interior cavity of the docking station 104 for charging the battery 118, disinfection of the intraoral device 102, data transfer from and/or to the intraoral device 102, and so forth. The docking station 104 includes a housing 300, a hinged door 302, a battery status indicator 304, and a disinfection status indicator 306. The door 302 is hingeably attached to the housing 300 to enable the door 302 to be opened and closed relative to the housing 300 for insertion and removal of the intraoral device 102 relative to the docking station 104. The battery status indicator 304 is operable to indicate a charging status of the battery 118 of the intraoral device 102. The battery status indicator 304, for instance, represents a light (e.g., LED light) that can be illuminated with different colors that represent different charging status of the battery 118. The disinfection status indicator 306 is operable to indicate a disinfection status of the intraoral device 102. The disinfection status indicator 306, for instance, represents a light (e.g., LED light) that can be illuminated with different colors that represent different disinfection status of the intraoral device 102, such as whether a preset disinfection cycled of the docking station 104 is in progress or complete.

FIG. 3b depicts the docking station 104 with the door 302 in an open position relative to the housing 300. The intraoral device 102 is positioned within a tray 308 positioned within a cavity 310 in the housing 300. Also positioned within the cavity 310 are the disinfection module 128 and a fan 312. The disinfection module 128, for instance, includes a UV light positioned to project UV light waves onto the intraoral device 102 for purpose of disinfecting the intraoral device 102. The fan 312 can circulate air within the cavity 310 to dry the intraoral device 102. FIG. 3b also depicts the tray 308 removed from the housing 300 of the docking station 104. Removal of the tray 308 enables cleaning, maintenance, and replacement of the tray 308.

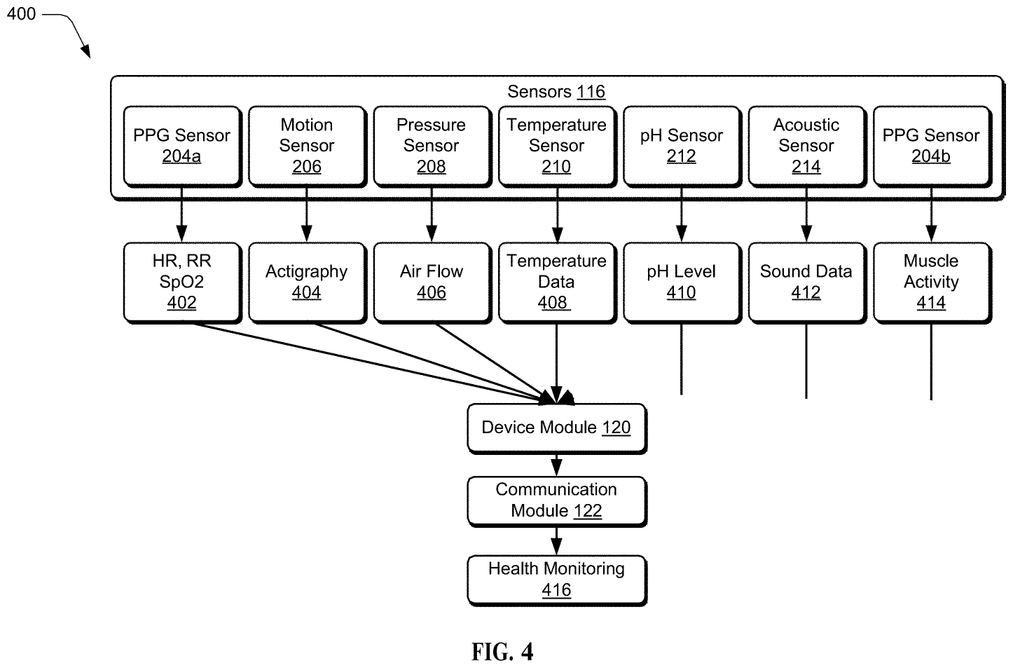

FIG. 4 depicts a system 400 for implementing aspects of systems and methods for intraoral monitoring. The system 400 includes the sensors 116 described above. For instance, the PPG sensor 204a captures sensor data 402 including heart rate, respiratory rate, and SpO2. The motion sensor 206 captures actigraphy data 404; the pressure sensor 208 captures air flow 406; the temperature sensor 210 captures temperature data 408; the pH sensor 212 captures pH level 410; the acoustic sensor 214 captures sound data 412; and the PPG sensor 204b captures muscle activity data 414. While not shown explicitly, the system 400 also includes a dielectric sensor configured for analysis of saliva and/or measuring tongue position of the user during sleep. The device module 120 receives this various data and leverages the communication module 122 to communicate the sensor data to various entities for performing health monitoring 416, such as for sleep stage monitoring, sleep apnea detection, AHI measurement, and so forth.

In at least some implementations, plurality of the sensors 116 may utilize different analog front-end circuitry, depending on the type of output signal. The output signals of these sensors 116 can be read by the device module 120 (e.g., a microcontroller) and then sent to another device (e.g., the client device 106) via the communication module 122. If a connection between the intraoral device 102 and another device is interrupted, the sensor data can be temporary stored in a memory of the device module 120 and/or in an on-board flash memory. Once a stable connection is re-established between the intraoral device 102 and a receiving station, real-time data transmission can be resumed.

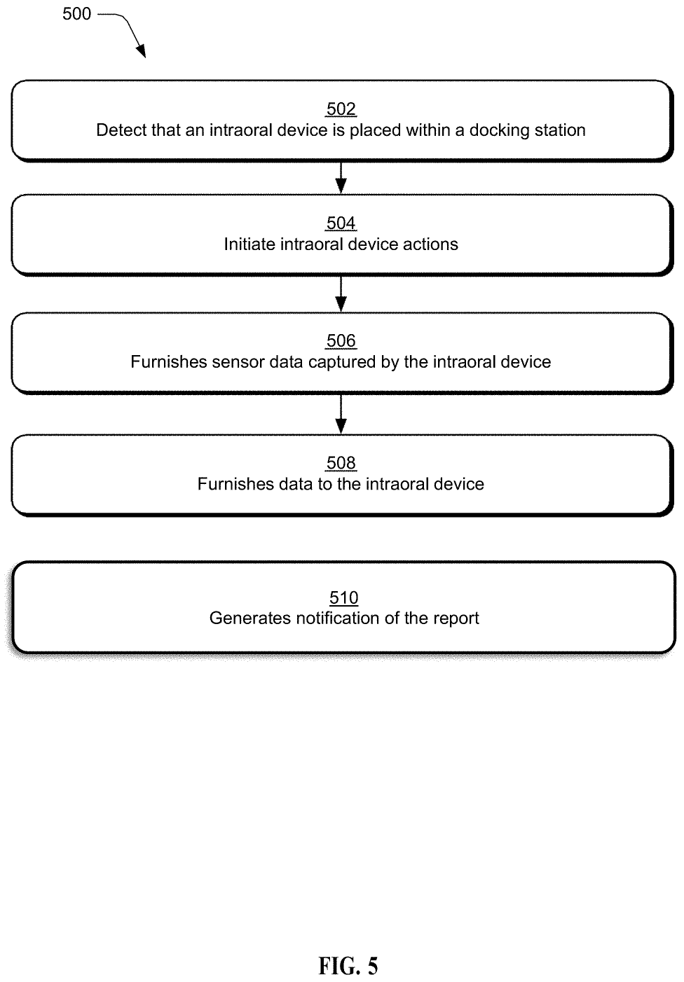

FIG. 5 depicts an example method 500 for intraoral monitoring and utilizing sensor data obtained as part of intraoral monitoring in accordance with one or more implementations. The method 500, for instance, is performed at least in part in the context of environment 100.

Step 502 detects that an intraoral device is placed within a docking station 104. A user, for instance, places the intraoral device 102 within the docking station 104 and latches the door 302. The docking station 104 can transmit a signal in response to the door 302 being closed, such as to the monitor app 132.

Step 504 initiates intraoral device actions. For instance, in response to the intraoral device 102 being placed into the docking station 104 and the door 302 being latched closed, the data module 126 of the docking station 104 initiates actions such as battery charging, cleaning, sensor data transmission, and so forth. Further, the DVMP 108 is notified of the locking event and in response requests sensor data from the data module 126.

Step 506 furnishes sensor data captured by the intraoral device. The docking station 104, for instance, furnishes the data to the DVMP 108 in an ordered fashion such as to preserve battery power, allow post-processing of information in the appropriate location, and so forth. In at least one implementation the DVMP 108 may implement an algorithm that updates the data to remove false positives that muscle activity has occurred, such as by comparing accelerometer data to PPG data. The updated data may be stored in a separate node such as at the DMP 110. Further, the DMP 110 can furnish the data to the RMS 112 and notify clinician and/or other personnel of updated data, trends, and other extracted insights.

In at least one implementation, the plurality of sensors 116 is configured to couple to the device module 120. The plurality of sensors 116 is further configured to transmit sensed signals associated with health parameters to the device module 120, in real time, using wireless communication devices. The device module 120 includes memory, controller and segregation module. In an embodiment, memory is communicatively coupled to a database (not shown in figures) which is configured to store various health parameters and their respective ranges, various health reports of the user, and basic details of all users. If a connection between the intraoral device 102 and user device is interrupted, the sensor data can be temporary stored in a memory of the device module 120 and/or in an on-board flash memory. Once a stable connection is re-established between the intraoral device 102 and a receiving station, real-time data transmission can be resumed.

The controller is configured to receive the sensed signals of the health parameters associated with the user from the plurality of sensors 116. The controller is configured to couple with both the memory and the segregation module, and is further configured to generate and transmit sensed data of the sensed signals of the health parameters associated with the user to the memory.

The segregation module is configured to segregate the sensed data of the health parameters associated with the user. The segregation module is further configured to cooperate with the memory to compare the sensed data with the stored predetermined threshold values and data and ranges corresponding to the plurality of health parameters associated with the users. For instance, the plurality of health parameters such as peripheral oxygen, temperature, pressure, motion, sound, pH, apnea hypopnea index (AHI) is segregated using various machine learning techniques.

In an embodiment, the segregation module is also configured to analyze the real time sensed data by employing machine learning techniques such as Gradient boosted techniques, Decision tree techniques and Logistic regression techniques to evaluate emergency admission cases with prediction and analysis using Information Communication Technology (ICT) techniques.

Step 508 furnishes data to the intraoral device 102. The DVMP 108, for instance, furnishes data to the intraoral device 102 via the docking station 104, such as for authentication requests, firmware updates, and so forth. In at least one implementation the DVMP 108 may send updated data generated at step 506 to the intraoral device 102 in order to execute a calibration activity that accounts for previous data errors and/or adjusts the positioning of a sensor to be more accurate, etc.

Step 510 generates a notification of the report. The controller is configured to receive the compared and analyzed data from the device module 120 and is configured to generate and transmit various types of notifications to the healthcare service provider and/or user based on the compared and analyzed data. The RMS 112, for instance, determines that the report is available and can generate and communicate a notification of the report, such as to clinician and/or other personnel. Accordingly, during an appointment with the user (or anytime) a clinician may review the report and provide useful information to that user.

In at least one implementation, the controller is configured to employ energy efficient FoG based IoT network techniques to monitor user's health conditions. In another embodiment, the controller can be implemented as one or more microprocessors, microcomputers, microcontrollers, digital signal processors, central processing units, state machines, logic circuitries, and/or any devices that manipulate signals based on operational instructions.

Further, the communication module 122 is configured to transmit the data to the cloud based remote server for historical data storage and monitoring log via a network. In an embodiment, the network 114 may include the Internet, wireless network, wired network, one or more telecommunications networks (e.g., Public Switched Telephone Networks (PSTNs)), a wired or wireless network, a wireless area network, a Wireless Video Are Network (WVAN), a Local Area Network (LAN), a WLAN, a PAN, a WPAN, WANs, metropolitan area networks (MANs), or an intranet.

Security-Based Attributes

Generally, information collected by the intraoral device 102 is collected with consent from the unique user. When the intraoral device 102 is manufactured independently it is assigned an identifier, serial number. An intraoral device 102 that is paired with a unique user of the intraoral device 102. Each unique user gives consent for the present system to collect, transmit, analyze. Only Users with authorized permission(s) to collect, view, evaluate, share, distribute, and analyze unique User Data are allowed. Data transmission from any portion of the present system to another is encrypted throughout the entirety of its life within the present system.

In at least one implementation, block-chain techniques can be utilized as part of user data storage, transfer, and access, which can improve data security, privacy, and data accessibility of various users of the described systems. For instance, attributes of block-chain including cryptography, decentralization, and consensus, ensure trust in transactions that involve health-related data. Utilizing the described techniques, for example, user data is structured into blocks and each block contains a health-related transaction or bundle of transactions. Further, each new block connects to all the blocks before it in a cryptographic chain in such a way that greatly decreases the ability to tamper with the data. Health data-related transactions within the blocks can be validated and agreed upon by a consensus mechanism, which can ensure that each transaction is valid.

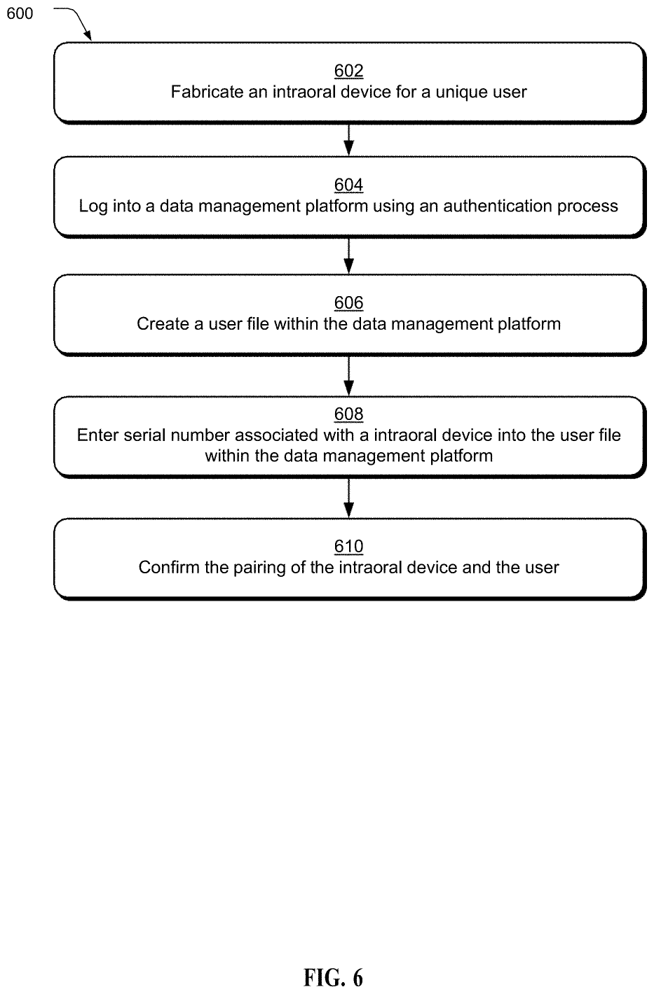

FIG. 6 depicts an example method 600 for security attributes as part of intraoral monitoring in accordance with one or more implementations. The method, for instance, is performed at least in part in the context of the environment 100 and can be implemented in conjunction with the methods 500.

Step 602 fabricates an intraoral device for a unique user. An authorized manufacturer, for instance, manufactures an intraoral device 102 for a specific user. Step 604 logs into a data management platform using an authentication process. The manufacturer, for instance, logs into the DMP 110. Step 606 creates a user file within the data management platform. The user file, for instance, includes a first name, last name, date of birth, and/or another unique user information. Step 608 enters the serial number associated with an intraoral device into a specific user file within the data management platform. Step 610 confirms the pairing of the intraoral device and the user. The manufacturer, for instance, confirms the pairing of the intraoral device and the user.

The example methods described above are performable in various ways, such as for implementing different aspects of the systems and scenarios described herein. Generally, any services, components, modules, methods, and/or operations described herein are able to be implemented using software, firmware, hardware (e.g., fixed logic circuitry), manual processing, or any combination thereof. Some operations of the described methods, for example, are described in the general context of executable instructions stored on computer-readable storage memory that is local and/or remote to a computer processing system, and implementations include software applications, programs, functions, and the like. Alternatively, or in addition, any of the functionalities described herein is performable, at least in part, by one or more hardware logic components, such as, and without limitation, Field-programmable Gate Arrays (FPGAs), Application-specific Integrated Circuits (ASICs), Application-specific Standard Products (ASSPs), System-on-a-chip systems (SoCs), Complex Programmable Logic Devices (CPLDs), and the like. The order in which the methods are described is not intended to be construed as a limitation, and any number or combination of the described method operations are able to be performed in any order to perform a method, or an alternate method.

Having described example procedures in accordance with one or more implementations, consider now some examples of sensor data analysis according to various techniques described herein.

FIG. 7 depicts graphs 700 that illustrates PPG signals at red and IR wavelengths measured via the intraoral device 102 in accordance with one or more implementations. A graph 700a includes an axis 702 that represents time (ms) and an axis 704 that represents intensity (arbitrary unit (a.u) Further, a graph 700b includes an axis 706 that represents time (ms) and an axis 708 that represents intensity (a.u).

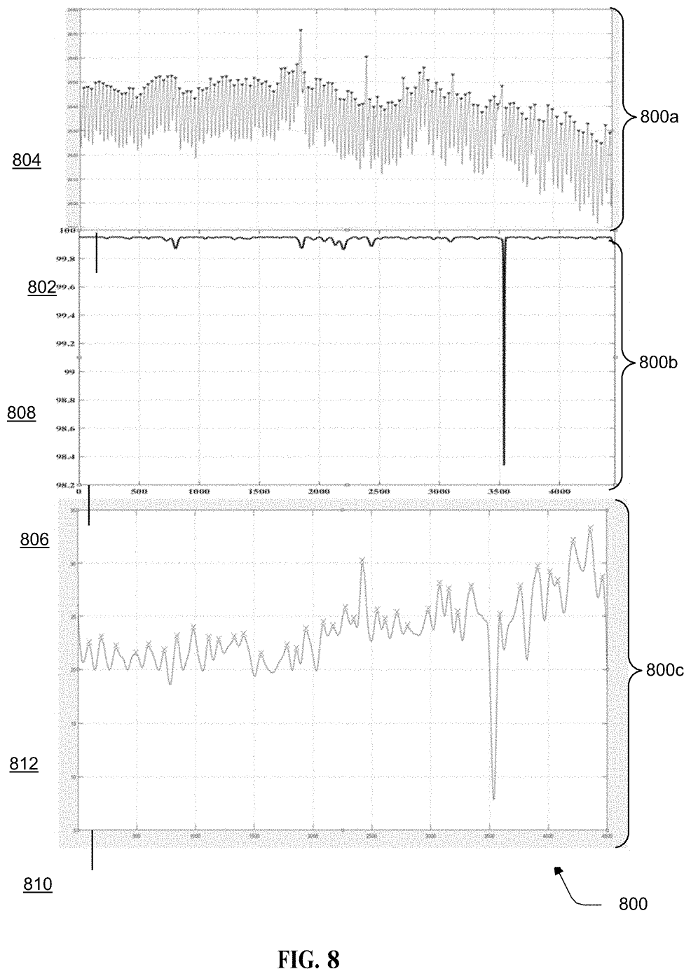

FIG. 8 depicts graphs 800 that illustrate estimated HR, SpO2, and RR from intraorally measured PPG signals measured via the intraoral device 102 in accordance with one or more implementations. For instance, a graph 800a includes an axis 802 that represents time (ms) and an axis 804 that represents intensity (a.u); a graph 800b includes an axis 806 that represents time (ms) and an axis 808 that represents SpO2 percentage; and a graph 800c includes an axis 810 that represents time (ms) and an axis 812 that represents normalized amplitude.

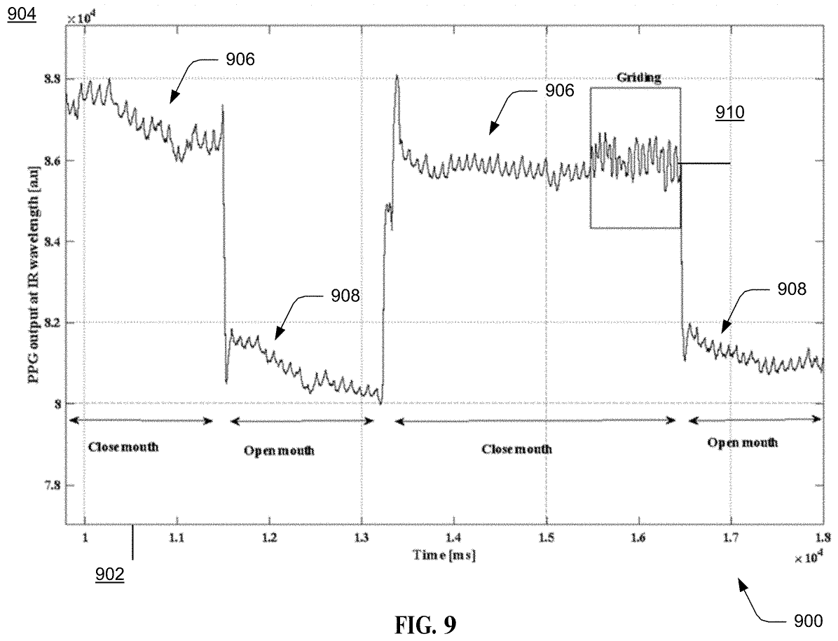

FIG. 9 depicts a graph 900 that illustrates intraorally monitoring masseter muscle activity by using a PPG sensor in accordance with one or more implementations. The graph 900 includes an axis 902 that represents time values and an axis 904 that represents values for PPG sensor output at an infrared (IR) wavelength. As depicted the graph 900 shows different PPG values over time for different masseter activities which can be correlated to different oral cavity states, such as closed mouth states 906 and open mouth states 908. Further, a grinding state 910 is depicted that indicates that a user is likely grinding their teeth, e.g., a possible sign of bruxism. These different oral cavity states can be correlated to different sleep states, such as to characterize sleep conditions at different times.

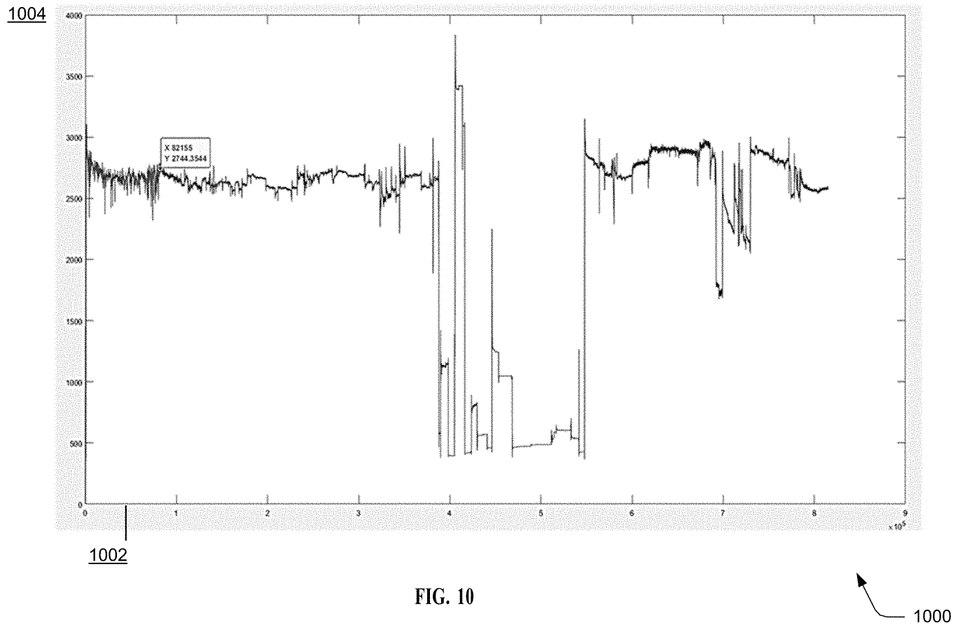

FIG. 10 depicts a graph 1000 that illustrates muscle activity measured by the intraoral device 102 in accordance with one or more implementations. The graph 1000 includes an axis 1002 that represents time (ms) and an axis 1004 that represents intensity (a.u).

Consider now an example system and device that are able to be utilized to implement the various techniques described herein.

### Example System and Device

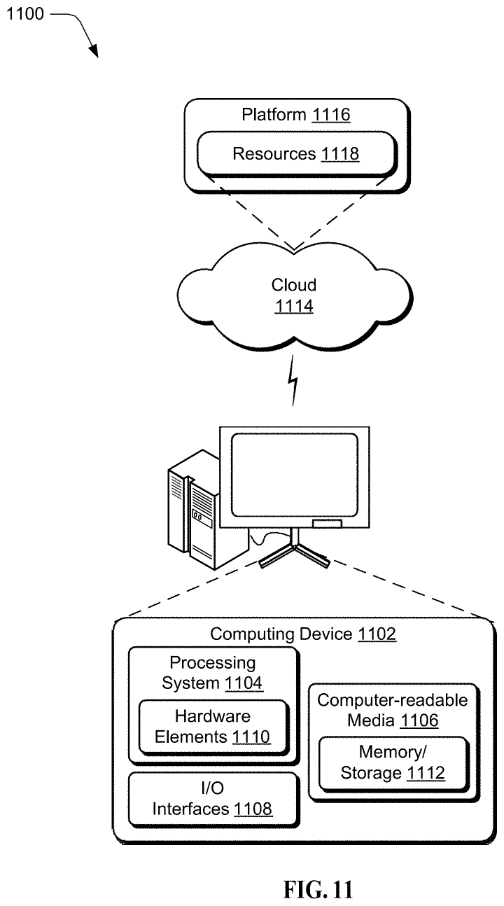

FIG. 11 illustrates an example system 1100 that includes an example computing device 1102 that is representative of one or more computing systems and/or devices that are usable to implement the various techniques described herein. The computing device 1102 includes, for example, a server of a service provider, a device associated with a client (e.g., a client device), an on-chip system, and/or any other suitable computing device or computing system.

The example computing device 1102 as illustrated includes a processing system 1104, one or more computer-readable media 1106, and one or more I/O interfaces 1108 that are communicatively coupled, one to another. Although not shown, the computing device 1102 further includes a system bus or other data and command transfer system that couples the various components, one to another. For example, a system bus includes any one or combination of different bus structures, such as a memory bus or memory controller, a peripheral bus, a universal serial bus, and/or a processor or local bus that utilizes any of a variety of bus architectures. A variety of other examples are also contemplated, such as control and data lines.

The processing system 1104 is representative of functionalities to perform one or more operations using hardware. Accordingly, the processing system 1104 is illustrated as including hardware elements 1110 that are be configured as processors, functional blocks, and so forth. This includes example implementations in hardware as an application specific integrated circuit or other logic device formed using one or more semiconductors. The hardware elements 1110 are not limited by the materials from which they are formed or the processing mechanisms employed therein. For example, processors are comprised of semiconductor(s) and/or transistors (e.g., electronic integrated circuits (ICs)). In such a context, processor-executable instructions are, for example, electronically-executable instructions.

The computer-readable media 1106 is illustrated as including memory/storage 1112. The memory/storage 1112 represents memory/storage capacity associated with one or more computer-readable media. In one example, the memory/storage component 1112 includes volatile media (such as random-access memory (RAM)) and/or nonvolatile media (such as read only memory (ROM), Flash memory, optical disks, magnetic disks, and so forth). In another example, the memory/storage component 1112 includes fixed media (e.g., RAM, ROM, a fixed hard drive, and so on) as well as removable media (e.g., Flash memory, a removable hard drive, an optical disc, and so forth). The computer-readable media 1106 is configurable in a variety of other ways as further described below.

Input/output interface(s) 1108 are representative of functionalities to allow a user to enter commands and information to computing device 1102, and also allow information to be presented to the user and/or other components or devices using various input/output devices. Examples of input devices include a keyboard, a cursor control device (e.g., a mouse), a microphone, a scanner, touch functionalities (e.g., capacitive or other sensors that are configured to detect physical touch), a camera (e.g., which employs visible or non-visible wavelengths such as infrared frequencies to recognize movement as gestures that do not involve touch), and so forth. Examples of output devices include a display device (e.g., a monitor or projector), speakers, a printer, a network card, tactile-response device, and so forth. Thus, the computing device 1102 is configurable in a variety of ways as further described below to support user interaction.

Various techniques are described herein in the general context of software, hardware elements, or program modules. Generally, such modules include routines, programs, objects, elements, components, data structures, and so forth that perform particular tasks or implement particular abstract data types. The terms “module,” “functionalities,” and “component” as used herein generally represent software, firmware, hardware, or a combination thereof. The features of the techniques described herein are platform-independent, meaning that the techniques are implementable on a variety of commercial computing platforms having a variety of processors.

Implementations of the described modules and techniques are storable on or transmitted across some form of computer-readable media. For example, the computer-readable media includes a variety of media that that is accessible to the computing device 1102. By way of example, and not limitation, computer-readable media includes “computer-readable storage media” and “computer-readable signal media.”

“Computer-readable storage media” refers to media and/or devices that enable persistent and/or non-transitory storage of information in contrast to mere signal transmission, carrier waves, or signals per se. Thus, computer-readable storage media refers to non-signal bearing media. The computer-readable storage media includes hardware such as volatile and non-volatile, removable, and non-removable media and/or storage devices implemented in a method or technology suitable for storage of information such as computer readable instructions, data structures, program modules, logic elements/circuits, or other data. Examples of computer-readable storage media include, but are not limited to, RAM, ROM, EEPROM, flash memory or other memory technology, CD-ROM, digital versatile disks (DVD) or other optical storage, hard disks, magnetic cassettes, magnetic tape, magnetic disk storage or other magnetic storage devices, or other storage device, tangible media, or article of manufacture suitable to store the desired information and which are accessible to a computer.

“Computer-readable signal media” refers to a signal-bearing medium that is configured to transmit instructions to the hardware of the computing device 1202, such as via a network. Signal media typically embodies computer readable instructions, data structures, program modules, or other data in a modulated data signal, such as carrier waves, data signals, or other transport mechanism. Signal media also include any information delivery media. The term “modulated data signal” means a signal that has one or more of its characteristics set or changed in such a manner as to encode information in the signal. By way of example, and not limitation, communication media include wired media such as a wired network or direct-wired connection, and wireless media such as acoustic, RF, infrared, and other wireless media.

As previously described, hardware elements 1110 and computer-readable media 1106 are representative of modules, programmable device logic and/or fixed device logic implemented in a hardware form that is employable in some embodiments to implement at least some aspects of the techniques described herein, such as to perform one or more instructions. Hardware includes components of an integrated circuit or on-chip system, an application-specific integrated circuit (ASIC), a field-programmable gate array (FPGA), a complex programmable logic device (CPLD), and other implementations in silicon or other hardware. In this context, hardware operates as a processing device that performs program tasks defined by instructions and/or logic embodied by the hardware as well as a hardware utilized to store instructions for execution, e.g., the computer-readable storage media described previously.

Combinations of the foregoing are also employable to implement various techniques described herein. Accordingly, software, hardware, or executable modules are implementable as one or more instructions and/or logic embodied on some form of computer-readable storage media and/or by one or more hardware elements 1110. For example, the computing device 1102 is configured to implement particular instructions and/or functions corresponding to the software and/or hardware modules. Accordingly, implementation of a module that is executable by the computing device 1102 as software is achieved at least partially in hardware, e.g., through use of computer-readable storage media and/or hardware elements 1110 of the processing system 1104. The instructions and/or functions are executable/operable by one or more articles of manufacture (for example, one or more computing devices 1102 and/or processing systems 1104 to implement techniques, modules, and examples described herein.

The techniques described herein are supportable by various configurations of the computing device 1102 and are not limited to the specific examples of the techniques described herein. These functionalities are also implementable entirely or partially through use of a distributed system, such as over a “cloud” 1114 as described below.

The cloud 1114 includes and/or is representative of a platform 1116 for resources 1118. The platform 1116 abstracts underlying functionalities of hardware (e.g., servers) and software resources of the cloud 1114. For example, the resources 1118 include applications and/or data that are utilized while computer processing is executed on servers that are remote from the computing device 1102. In some examples, the resources 1118 also include services provided over the Internet and/or through a subscriber network, such as a cellular or Wi-Fi network.

The platform 1116 abstracts the resources 1118 and functions to connect the computing device 1102 with other computing devices. In some examples, the platform 1116 also serves to abstract scaling of resources to provide a corresponding level of scale to encountered demand for the resources that are implemented via the platform. Accordingly, in an interconnected device embodiment, implementation of functionalities described herein is distributable throughout the system 1100. For example, the functionalities are implementable in part on the computing device 1102 as well as via the platform 1116 that abstracts the functionalities of the cloud 1114.

Technical Advancements

The present disclosure described herein above for an intraoral monitoring device has several technical advantages including, but not limited to, the realization of:
PPG signals from the PPG sensors are resistant to moisture;
the intraoral monitoring device can be placed inside the oral cavity of the user (e.g., the exterior of the maxilla (upper jaw) or mandible (lower jaw));
monitor one's health at their convenience;
portable and light weight;
easy to maintain;
user friendly;
improves patient safety through direct access to the medical history, treatments online;
provides timely, better and cheaper access to information;
works on portable and convenient source of power;
offers another option for people who cannot tolerate CPAP; and
reduce the labor burden for sleep clinicians who are currently outnumbered at 55,000:1 for Obstructive Sleep Apnea (OSA) patients.

The embodiments herein and the various features and advantageous details thereof are explained with reference to the non-limiting embodiments in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

The foregoing description of the specific embodiments so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

The use of the expression “at least” or “at least one” suggests the use of one or more elements or ingredients or quantities, as the use may be in the embodiment of the disclosure to achieve one or more of the desired objects or results.

Any discussion of documents, acts, materials, devices, articles or the like that has been included in this specification is solely for the purpose of providing a context for the disclosure. It is not to be taken as an admission that any or all of these matters form a part of the prior art base or were common general knowledge in the field relevant to the disclosure as it existed anywhere before the priority date of this application.

The numerical values mentioned for the various physical parameters, dimensions or quantities are only approximations and it is envisaged that the values higher/lower than the numerical values assigned to the parameters, dimensions or quantities fall within the scope of the disclosure, unless there is a statement in the specification specific to the contrary.

While considerable emphasis has been placed herein on the components and component parts of the preferred embodiments, it will be appreciated that many embodiments can be made and that many changes can be made in the preferred embodiments without departing from the principles of the disclosure. These and other changes in the preferred embodiment as well as other embodiments of the disclosure will be apparent to those skilled in the art from the disclosure herein, whereby it is to be distinctly understood that the foregoing descriptive matter is to be interpreted merely as illustrative of the disclosure and not as a limitation.

We claim:

1. An intraoral monitoring device comprising:
a flexible substrate;
a plurality of sensors embedded in a flexible substrate;
a rechargeable battery;
a device module configured to:
collect sensor data from the sensors;
store and transfer sensor data to other entities;
processing sensor data and transferring data to the intraoral device; and
a communication module for enabling data communication from and to the intraoral device.

2. The intraoral device as claimed in claim 1, wherein the flexible substrate is formed from a flexible material such as Kapton™ tape and/or other flexible material, such as a flexible polymer and is configured to be curved according to a curvature of a wearer.

3. The intraoral device as claimed in claim 1, wherein the battery comprises a single battery or an array of batteries.

4. The intraoral device as claimed in claim 1, wherein the plurality of sensors includes:
a plurality of PPG sensors configured to capture sensor data including heart rate, respiratory rate, SpO2, muscle activity and/or attributes of blood volume, such as in a microvascular bed of tissue;
a motion sensor configured to capture actigraphy data;
a pressure sensor configured to measure the intraoral pressure;
a temperature sensor configured to measure an oral cavity temperature as well as core housing temperature;
a pH sensor configured to measure the pH (e.g., relative acidity or alkalinity) within an oral cavity; and
an acoustic sensor configured to measure audio signals.

5. The plurality of sensors as claimed in claim 4, wherein the PPG sensors are positioned to measure muscle activity of a lateral pterygoid, buccinator and masseter muscle from inside an oral cavity.

6. The plurality of sensors as claimed in claim 4, wherein PPG signals from the PPG sensors are resistant to moisture.

7. The plurality of sensors as claimed in claim 4, wherein the acoustic sensor represents a low power microphone that is integrated into the substrate and is configured to measure a sound intensity in the oral cavity, such as the intensity of snoring from the oral cavity.

8. The intraoral device as claimed in claim 1, wherein the device module is configured to receive sensor data and leverages the communication module to communicate the sensor data to various entities to perform health monitoring, like sleep stage monitoring, sleep apnea detection, and AHI measurement.

9. The intraoral device as claimed in claim 1, wherein the communication module is configured to utilize different wireless and/or wired communication protocols, such as Bluetooth™, Near-field communication (NFC), Ultra-wideband (UWB), ZigBee etc.

10. A system for intraoral monitoring comprises:
an intraoral device;
a docking station configured to perform various functionalities for maintaining and interfacing with the intraoral device;
a client device configured to connect to the docking station and/or the intraoral device to provide various user functionalities;
a device management platform (DVMP) configured to provide data transfer between different entities of an environment and/or for processing of data received from the client device, e.g., sensor data generated at the intraoral device;
a data management platform (DMP); and
a remote monitor system (RMS).

11. The system for intraoral monitoring claimed in claim 10, wherein an intraoral device, docking station, client device, DVMP, DMP, and RMS are interconnectable via connectivity to a network and/or via direct device-to-device connectivity.

12. The system for intraoral monitoring claimed in claim 10, wherein the intraoral device is positioned within a tray which is further placed within a cavity in a housing.

13. The system for intraoral monitoring claimed in claim 10, wherein the docking station comprises:
a housing;
a hinged door attached to the housing;
a battery status indicator configured to indicate a charging status of the battery of the intraoral device; and
a disinfection status indicator configured to indicate a disinfection status of the intraoral device.

14. The system for intraoral monitoring as claimed in claim 10, wherein the client device includes a monitor application (“app”) which is configured to receive data that is generated by the intraoral device and is further configured to enable data transfer to the intraoral device.

15. A method for intraoral monitoring comprises:
plurality of sensors configured to sense a plurality of health parameters from an associated user, in real time, and is further configured to generate a plurality of sensed signals;
a device module configured to cooperate with the plurality of sensors to receive the sensed signals, the device module includes:
a memory configured to store sensed data from the plurality of sensors corresponding to the plurality of health parameters associated with the users;
a controller configured to generate sensed data;
a segregation module configured to segregate the data corresponding to the plurality of health parameters associated with the users;
a communication module configured to enable data communication to and from the intraoral device.

16. The method for intraoral monitoring as claimed in claim 15, wherein the method uses block-chain techniques as part of user data storage, transfer, and access, which can improve data security, privacy, and data accessibility of various users.

17. The method for intraoral monitoring as claimed in claim 15, wherein the device module is configured to analyze real time sensed data by employing machine learning techniques like Gradient boosted techniques, Decision tree techniques and Logistic regression techniques to extract and store the data corresponding to the plurality of health parameters associated with the users and is further configured to segregate data corresponding to the plurality of health parameters associated with the users using Information Communication Technology (ICT) techniques.

18. The method for intraoral monitoring as claimed in claim 15, wherein the device module is configured to employ energy efficient FoG based IoT network techniques to monitor a user's health conditions.

19. The method for intraoral monitoring as claimed in claim 15, wherein the communication unit cooperates with the device module to transmit the data to a cloud—based server for data storage and monitoring using a network.

20. The method for intraoral monitoring as claimed in claim 15, the device module can be implemented as one or more microprocessors, microcomputers, microcontrollers, digital signal processors, central processing units, state machines, logic circuitries, and/or any devices that manipulate signals based on operational instructions.
